(19) United States
(12) Patent Application Publication
(10) Pub. No.: US 2025/0161651 A1
(43) Pub. Date: May 22, 2025

(54) STERILE FLUIDIC CONNECTOR WITH RECONNECTABLE CONNECTORS AND METHOD OF ASEPTIC CONNECTION

(71) Applicant: EMD Millipore Corporation, Burlington, MA (US)

(72) Inventors: James Ormond, Belmont, MA (US); Robert Langlois, Groton, MA (US); Conor King, Marblehead, MA (US)

(21) Appl. No.: 18/724,974

(22) PCT Filed: Dec. 27, 2022
(86) PCT No.: PCT/US2022/054065

§ 371 Date: Jun. 27, 2024

Related U.S. Application Data
(60) Provisional application No. 63/298,390, filed on Jan. 11, 2022.

(51) Int. Cl. A61M 39/18; A61M 39/22

(52) U.S. Cl. CPC A61M 39/18; A61M 39/22

(57) ABSTRACT

A fluid transfer device comprising: a housing; a core positioned in the housing for relative movement with the housing, the core having a bore having an inlet and an outlet spaced from the inlet, and first and second gate-receiving regions spaced from the inlet and the outlet; and an actuator operatively connected in the device to cause relative movement between said core and said housing. Also disclosed is a method of creating an aseptic connection using the fluid transfer device.

This application claims priority of U.S. Provisional Application Ser. No. 63/298,390 filed Jan. 11, 2022, the disclosure of which is incorporated herein by reference.

### BACKGROUND

This disclosure relates to a fluid transfer device, especially for fluid transfer in bioprocessing of biological fluids. More particularly, embodiments of the disclosure relate to fluid transfer devices, such as connectors, that can be connected and disconnected to systems while maintaining a leak-free state and sterile, and methods of aseptic connection.

Biological processes require the transfer of media, such as liquids, into or from a container or the like. For example, tubing and valves, which enable fluid transfer between two systems, are typically employed. Such systems can be batch processes or semi-continuous processes. Continuous processing is much more complex than batch or semi-continuous processing.

When conducting complex fluid processes within a “closed” fluid system, it is often necessary to connect or link unit operations of a manufacturing process or to monitor the progress of the process. Also, it is often desirable to transfer the fluid without disturbing the process, such as may occur upon “opening” the receptacle or unit operation. For example, in the study and/or manufacture of biochemical products (e.g., biopharmaceuticals), a biochemical fluid is often contained in an aseptically “closed” fermenting tank, bioreactor, or like fluid container, wherein the fluid is processed over comparatively long periods of time, i.e., several days to weeks. By withdrawing and analyzing samples of the fluid intermittently in the course of the process, which is under diverse and changing chemical and environmental conditions, one can learn more about the progress of the process, and if called for, take prophylactic measures to change the outcome thereof. There also exists a need to connect multiple unit operations together or draw from or add to multiple vessels into a common flow conduit in a sterile manner without “opening” the vessel or flow conduit and disrupting the process, and which can subject the fluid(s) to contamination.

Similar problems arise when fluid is transported through a conduit, or a pipe, or other like fluid receptacle. Sampling of the fluid is often difficult because in many industrial systems, said receptacles are not easily opened or disassembled to allow one to withdraw fluid samples, especially in a sterile manner. While several fluid sampling techniques are known, certain technical issues can be noted. For example, certain integrated fluid sampling fixtures comprise stainless steel valves and piping which, for biopharmaceutical applications, often require laborious steam sterilization and cleaning prior to use. Other fluid sampling devices are difficult to integrate into extant fluid processing systems, for example, by requiring the installation of custom-fitted ports onto a host fluid receptacle. Still other devices, although adapted for use in standard industrial ports, are complex and costly instruments comprising valves, inlets, outlets, seals, needles, and other components, all precisely arranged, but capable of only one aseptic sample per sterilization cycle. Finally, the majority of fluid sampling devices, as is the case in many of those above, require in their operation the piercing of a septum using a hypodermic needle. Other systems use dip tubes, which cause contamination.

Biological processing of fluids requires the introduction and/or removal of materials from a process fluid stream in order to add components, such as media or buffers to a bioreactor; withdraw samples from the process fluid stream to check for microbial contamination, quality control, process control, etc., conduct unit operations such as mixing, filtration, cell culture, etc., and to fill the product into its final container such as vials, syringes, sealed boxes, bottles, single use storage containers such as film bags, single use mix bags/mixers, and the like.

Aseptic coupling devices can be used to connect two or more sterilized pathways. For example, aseptic coupling devices can be used to couple a fluid pathway from a first piece of processing equipment or container to a fluid pathway from a second piece of processing equipment or container to establish a sterile pathway for fluid transfer therebetween.

Typical aseptic coupling devices require a “dry-to-dry” or “dry connection” that is created using one or more pathway clamping devices placed upstream of the aseptic coupling devices so that the aseptic coupling devices are kept free of fluid while the connection between the aseptic coupling devices is made. Once the sterile connection between the aseptic coupling devices is made, the clamping devices are removed to allow fluid to flow through the aseptic coupling devices.

In view of the foregoing, a fluid transfer device that can provide a sterile wet connection, under pressure, provide a dripless or leak-free disconnection, and/or completely reverse the connection while maintaining a sterile flow path, represents an advance in the art.

### SUMMARY

Embodiments disclosed herein provide a fluid transfer device permitting “wet” connections, which may be under pressure, wherein the connection can be reversed, while leaving the connectors sterile and reusable. In some embodiments, the fluid transfer device is in the form of a connector or valve. In some embodiments, fluid lines are sealed off during the disconnect and reconnect process. In some embodiments and processes, the fluid transfer device is re-sealed with a gate for each disconnect/reconnect cycle to minimize drippage. In some embodiments, the fluid transfer device is a single-use device or includes single-use components. In some embodiments, the connectors attachable to the fluid transfer device may be re-used.

Some embodiments of the disclosure comprise a fluid transfer device enabling sterile fluid connections. Formation of a sterile connection includes attaching a first coupling member or connector to an inner valve core, displacing a gate from the first coupling member or connector to enable fluid communication between the first coupling member or connector and the core; after fluid transfer introducing a substitute or stored gate in the core into the first coupling member or connector, and decoupling the first coupling member or connector from the core. In some embodiments, the fluid communication is established though an internal fluid channel or bore in the core. In some embodiments, a second coupling member or connector may be attached to the core, a gate from the second coupling member or connector is displaced from the second coupling member or connector to enable fluid communication between the second coupling member or connector and the core; after fluid transfer introducing a substitute or stored gate in the core into the second coupling member or connector, and decoupling the second coupling member or connector from the core. In some embodiments, the fluid communication between the core and the second coupling member or connector is established though said fluid channel or bore in the core. In some embodiments, the fluid transfer is enabled by fluid communication between the first and second connectors through the bore. The various coupling members or connectors are preferably aseptic coupling members.

In some embodiments, the core is contained in a housing, which may be formed of first and second housing members that are engageable and lockable together, such as by fastening at least one actuator to the core through the housing. Alternatively, the housing may be a single integral unit. First and second substitute or stored gates may be positioned within the housing, such as in gate-receiving regions of the core. The core is contained within the housing and has a top face and a bottom face opposite the top face. In certain embodiments, the at least one actuator may be keyed to the top face or to the bottom face. Actuation of the at least one actuator causes relative movement between the core and the housing. In some embodiments, the movement is rotational. In some embodiments, the movement is rotational about an axis perpendicular to the top and bottom faces of the core. In some embodiments, the movement is linear.

In the assembled condition, the housing has a pair of spaced openings or gaps each configured to receive, in locking engagement, one of the first and second coupling members or connectors 12, 14. Each of the first and second coupling members or connectors may be attached or attachable to tubing or the like, which may be placed in fluid communication with a component capable of a unit operation, such as a filtration or chromatography device, a bioreactor, a vessel, etc. Each of the connectors is configured to hold a respective displaceable gate in sealing relation with the connector. When a connector is coupled to the housing, the displaceable gate in each connector is displaced from its connector by actuation of the device such as relative rotation or translation of the core and housing, which also places the core in a fluid transfer position. In this position, a fluid channel or bore within the core aligns with the connectors and allows for fluid communication between the connectors through the fluid channel or bore. One of the spaced openings may be a fluid inlet into the fluid transfer device, and the other a fluid outlet from the fluid transfer device.

A first substitute or stored gate may be positioned in the housing such that upon further appropriate actuation of the core (e.g., actuation moving the bore out of fluid communication with the first and second connectors), the first stored gate is moved to a position in sealing relation with the first connector. Similarly, a second substitute or stored gate may be positioned in the housing such that upon appropriate actuation of the core (e.g., actuation moving the bore out of fluid communication with the first and second connectors), the second stored gate moves to a position in sealing relation with the second connector. The movement of the first and second substitute or stored gates into their respective positions in sealing relation with their respective connectors may be simultaneous or substantially simultaneous. The first and second connectors may then be decoupled from the housing, each carrying its substitute or stored gate, in a dripless manner.

Accordingly, in some embodiments the fluid transfer device is valve actuatable between an open position allowing fluid transfer from the first connector through the bore to the second connector, and a closed position preventing fluid communication between the first and second connectors. In some embodiments, actuation of the valve from the closed position to the open position aligns the bore with the first and second connectors. In some embodiments, actuation of the valve from the closed position to the open position displaces a gate in each of the connectors, thereby allowing fluid access to and/or from each of the connectors through the aligned bore. In some embodiments, upon completion of the fluid transfer, the valve may be further actuated to a closed positioned to move the bore out of alignment with the first and second connectors, and introduce respective substitute gates into each of the connectors. The connectors (and their respective substitute gates) then may be decoupled from the valve in a dripless manner. In some embodiments, the valve is a rotary valve whereby actuation of the valve is caused by applying a rotational force. In some embodiments, each of the rotary actuations (e.g., from the closed position to the open fluid transfer position, and then to a closed position) is in the same direction, e.g., clockwise or counter-clockwise. In certain embodiments, the valve is a linear valve whereby actuation of the valve is causes linear movement of the core relative to the housing.

Also disclosed is a method of creating an aseptic connection, comprising coupling a first coupling member or connector and a second coupling member or connector to a valve core, the valve core having an internal or inner bore, first and second gate-receiving regions and first and second gates respectively occupying the first and second gate-receiving regions; actuating the valve core to establish fluid communication between the first and second connectors through the inner bore; and upon completion of fluid transfer, further actuating the valve core to move the first and second gates out of the first and second gate-receiving regions respective sealing engagement with the first and second coupling members or connectors. In some embodiments, the first and second connectors may be removed from the valve core after the first and second gates are sealingly engaged with their respective connectors.

In some embodiments, the valve core comprises first and second radially outwardly protruding regions, and actuation of the valve core to establish fluid communication between the first and second connectors through the inner bore causes the first and second radially outwardly protruding regions to respectively receive the first and second connectors.

In some embodiments, the first and second connectors each hold a displaceable gate when the first and second connectors are coupled to the valve core, and actuation of the valve core to establish fluid communication between the first and second connectors through the inner bore causes the first and second radially outwardly protruding regions to respectively displace the first and second displaceable gates. The first and second displaceable gates are housed in gate-receiving regions of the core.

These and other embodiments, and provisions thereof, will become clear from the description, claims, and figures below. Various benefits, aspects, novel and inventive features of the present disclosure, as well as details of exemplary embodiments thereof, will be more fully understood from the following description and drawings. So the manner in which the features disclosed herein can be understood in detail, more particular descriptions of the embodiments of the disclosure, briefly summarized above, may be had by reference to the appended drawings. It is to be noted, however, that the appended drawings illustrate only some typical embodiments of this disclosure and are therefore not to be considered limiting of its scope, for the described embodiments may admit to other equally effective probe and sensor chambers. It is also to be understood that elements and features of one embodiment may be found in other embodiments without further recitation and that, where possible, identical reference numerals have been used to indicate comparable elements that are common to the figures.

As used herein, the singular forms “a,” “an,” and “the” include plural referents unless the context clearly dictates otherwise. Unless defined otherwise, all technical and scientific terms used herein have the same meaning as commonly understood by one of ordinary skill in the art to which these embodiments pertain.

### BRIEF DESCRIPTION OF THE DRAWINGS

FIG. 1 is a perspective view of a fluid transfer device according to embodiments of the disclosure;

FIGS. 2A, 2B, 2C and 2D depict perspective views of a barbed fitting according to embodiments of the disclosure;

FIG. 3 is an exploded view of a fluid transfer device and barbed fittings according to embodiments of the disclosure;

FIG. 4A is a perspective view of a core according to embodiments of the disclosure;

FIG. 4B is a top view of the core according to embodiments of the disclosure;

FIG. 4C is a bottom view of the core according to embodiments of the disclosure;

FIG. 5 is a perspective view, in cross-section, of a fluid transfer device with barb fittings attached, according to embodiments of the disclosure;

FIG. 6 is a front view, in cross-section, of a fluid transfer device with barb fittings attached, according to embodiments of the disclosure;

FIG. 7 is an exploded view of a linear embodiment of a fluid transfer device in accordance with certain embodiments;

FIG. 8 is a perspective view, in cross-section, of the linear embodiment of FIG. 7;

FIGS. 9A, 9B, 9C, 9D and 9E are respective perspective views, in cross-section, of certain positions of the fluid transfer device of FIG. 7;

FIG. 10 is a perspective view of a connector and gate in accordance with the embodiment of FIG. 7; and

FIG. 11 is a perspective view, in cross-section, of a fluid transfer device shown with barb fittings attached, according to an alternative embodiment of the disclosure.

### DETAILED DESCRIPTION

In some embodiments, the fluid transfer device includes first and second housing members, which can be engaged or coupled and locked together to form a housing. In the assembled and engaged or coupled state, sterile fluid communication through the two housing members may be created, and thus between valve members carried or carriable by the two members. In certain embodiments, fluid transfer though the housing may be effectuated by actuation of the device. In certain embodiments, the actuation is carried out by relative movement of a valve core contained in the housing and the housing. In certain embodiments, the relative movement includes rotational movement. In certain embodiments, the relative movement includes linear movement. In certain embodiments, the linear movement includes moving the core and the housing in opposing directions.

Turning now to FIG. 1, there is shown an embodiment of a fluid transfer device 10. In the embodiment shown, the fluid transfer device 10 includes a first coupling member or connector 12 shown coupled to a core 13, and a second coupling member or connector 14 shown coupled to the core 13. Each of the first and second coupling members 12 and 14 may be attached to or integral to a barbed fitting 15 or tri-clover fitting or the like, configured to connect to suitable tubing or the like in a conventional and well-known manner. In the embodiment shown, the fluid transfer device 10 includes a first actuator 16 and a second actuator 17 spaced from the first actuator 16. Each of the actuators 16, 17 may be attached to the core 13 by a fastener or the like, in a manner that allows relative movement of the actuators and the core 13, such as rotational movement.

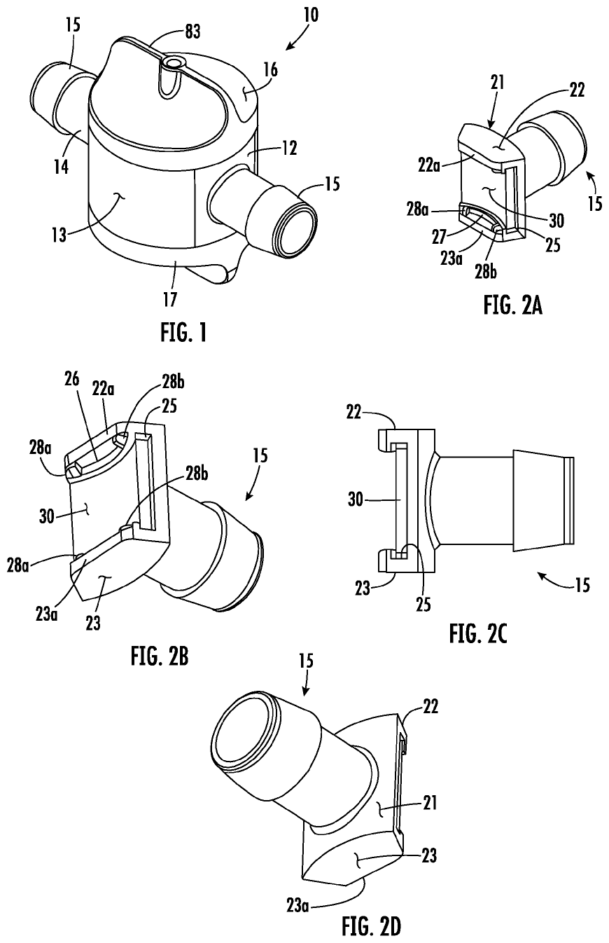

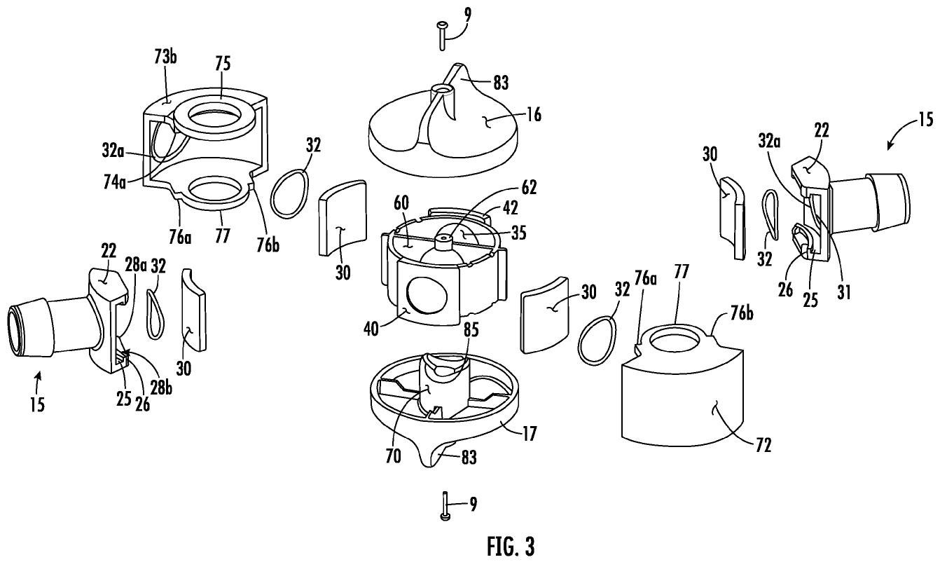

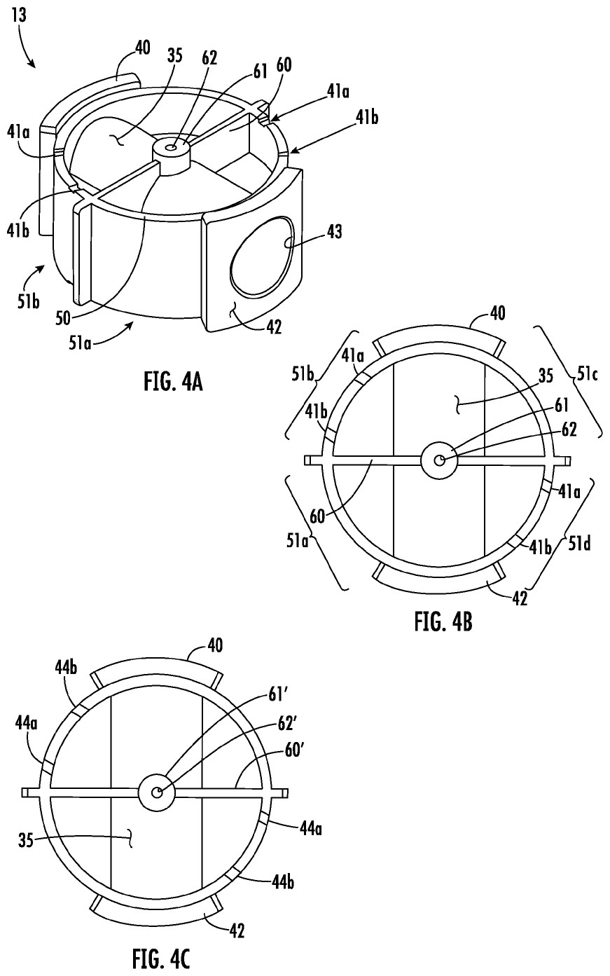

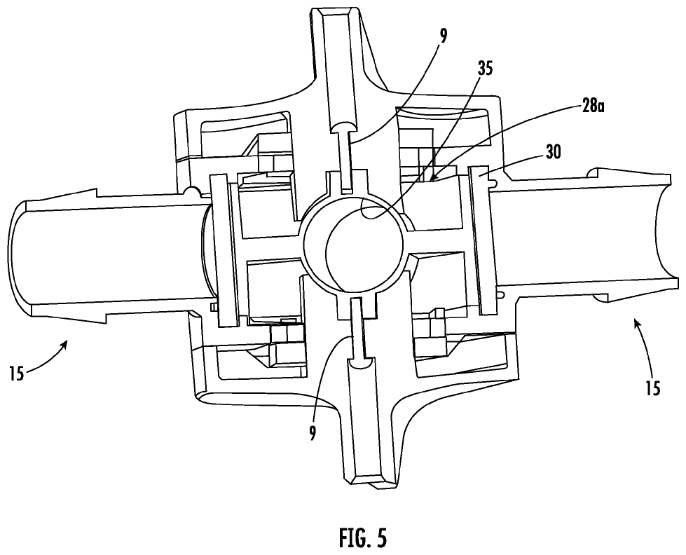

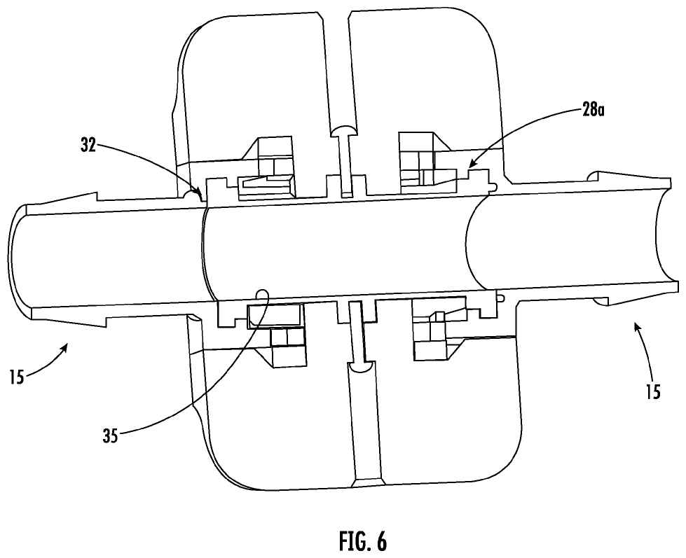

FIGS. 2A, 2B, 2C and 2D illustrate an embodiment of the first and second coupling members or connectors 12, 14, which may be identical. As can be best seen in FIGS. 2A and 2D, the coupling member 12, 14 includes an arcuate outer surface 21, a top arm 22 and a bottom arm 23 each extending in the same direction from the arcuate outer surface 21, each of which tapers to a respective free end 22a, 23a. The underside of each arm 22, 23 has features thereon so as to form, in cooperation with the backside of the arcuate outer surface 21, an arcuate groove 25 configured to slidingly receive a displaceable gate 30. The radius of curvature of the groove 25 matches the radius of curvature of the gate 30. In some embodiments, each of the features forming the groove 25 in cooperation with the backside of the arcuate outer surface 21 is an arcuate ridge or protrusion 26, 27 respectively extending from the underside of the arm 22 and 23. Each of the arcuate ridges or protrusions 26, 27 on each coupling member terminates in a respective free end defining its height, and the distance between the respective free ends on a coupling member is sufficient to receive and engage with the core 13, as discussed in greater detail below. The underside of each arm 22, 23 also includes two spaced tabs 28a, 28b that are configured to be received in corresponding spaced slots 41a, 41b and 44a, 44b (FIG. 4A) on the core 13 to facilitate detachment of the first and second coupling members 12, 14 from the core 13. In some embodiments the tabs 28a, 28b are ramped and their plasticity allows them to flex to catch or snap over the top and bottom rim of the outer circumferential face 50 to attach and retain the connector to the core and ultimately be in fluid communication with the bore 35 when in the fluid transfer position where the flow path is opened (FIGS. 5 and 6). As can be seen in FIG. 3, each coupling member 12, 14 has a through hole 31, and a groove 32a for receiving a seal member 32 such as a gasket or O-ring to help seal a gate 30 against the through hole 31 when the gate is positioned in the groove 25, and to help seal the bore 35 against the through hole 31 when the device is in the fluid transfer position. In some embodiments, each of the coupling members is symmetrical about a horizontal axis, and thus can be attached to the fluid transfer device in the orientation shown in FIG. 2A, or in an up-side-down orientation to that shown in FIG. 2A, i.e., rotated 180°.

In some embodiments, the core 13 is generally cylindrical or disk-shaped as best seen in FIGS. 3, 4A, 4B and 4C. It includes a preferably central fluid channel or bore 35 that extends through the core 13 to provide fluid communication between the first and second coupling members 12, 14 when the first and second coupling members are sealingly engaged with the core 13 and the bore 35 is appropriately aligned (e.g., the bore 35 is in the fluid transfer position). In certain embodiments, the outer circumferential face 50 of the core 13 includes a first radially outwardly extending region 40 and a second radially outwardly extending region 42. Each of the first and second radially outwardly extending regions 40, 42 has a radius of curvature that matches the radius of curvature of a groove 25, and includes a respective through hole 43 that communicates with a respective end of the fluid channel or bore 35. Preferably the through holes 43 are spaced 180° from each other. A portion of each of the first and second radially outwardly extending regions 40, 42 extends axially above and below the outer circumferential face of the core 13 (FIG. 4A) and is configured to be slidingly received in a respective arcuate groove 25 of the first or second coupling member 12, 14. In some embodiments, each of the first and second radially outwardly extending regions 40, 42 has the same or similar dimensions as a gate 30 (except for the presence of the through hole 43).

In some embodiments, the top surface of the outer circumferential face 50 of the core 13 has two pairs of spaced slots 41a, 41b, and the bottom surface of the outer circumferential face 50 also has two pairs of spaced slots 44a, 44b. Preferably each slot 41a is positioned directly above each slot 44a, and each slot 41b is positioned directly above each slot 44b. The slots are configured and positioned to release corresponding tabs 28a, 28b of the first and second coupling members 12, 14 to assist in decoupling the first and second coupling members from the core 13 such as after fluid transfer has been completed. In certain embodiments, the first and second coupling members are coupled to the core 13 with respective gates 30 positioned in place in the coupling members, occupying the grooves 25. As the device is actuated and the first and second coupling members are moved into alignment with the bore 35, the first and second radially outwardly extending regions 40, 42 obstruct and thereby prevent the gates 30 that are respectively positioned in the first and second coupling members from moving with the coupling members, and thus these gates 30 remain in respective gate-receiving regions of the core 13 while the coupling members move into fluid communication with the bore 35 where they respectively engage with the first and second radially outwardly extending regions 40, 42. The first and second coupling members are now in the fluid transfer position, and are devoid of displaceable gates 30. The gates 30 so displaced preferably have the same radius of curvature as the gate-receiving regions 51b, 51d, remain in respective gate-receiving regions 51b, 51d of the core 13 and may be ultimately disposed of with the device.

FIGS. 4A, 4B and 4C illustrate an embodiment of features on the core 13 that receive actuators 16, 17. In the embodiment shown in FIG. 4B, rib 60 is formed as a diameter of the top of the core 13, and includes a central cylindrical nub 61 having an aperture 62 configured to receive fastener 9 that secures actuator 16 to the core 13. Similarly, FIG. 4C illustrates rib 60′ formed as a diameter of bottom of the core 13, and includes a central cylindrical nub 61′ having an aperture 62′ configured to receive fastener 9 that secures actuator 17 to the core 13. Each rib 60, 60′ extends radially beyond the outer circumferential face 50, effectively dividing the outer circumferential face 50 into four equally dimensioned gate-receiving regions 51a, 51b, 51c and 51d.

In some embodiments, the underside of actuators 16, 17 each include an axially extending hub 70 that terminates in a free end that is shaped to mate with the outer surface of bore 35. Thus, the radius of curvature of the hub free end corresponds to the radius of curvature of the bore 35. Each hub free end includes a slot 85 dimensioned to receive a respective rib 60, 60′, which functions to key each actuator 16, 17 to the core 13. Each hub 70 includes an aperture 71 that receives a fastener 9 to capture the housing members 70 and secure the actuator to the core 13. Each actuator 16, 17 may have a handle or grip 83 extending from its surface to facilitate actuation by a user.

FIG. 3 illustrates an embodiment of the housing that contains the core 13. In the embodiment shown, the housing includes two identical generally arcuate housing members 70. Each generally arcuate housing member 70 includes an arcuate wall 72, and first and second spaced arms 73a, 73b that extend radially respectively from the top and bottom of the arcuate wall 72. Preferably one of the first or second arms 73a, 73b has a partial member 74 terminating in two spaced free ends 74a, 74b (only 74a shown) and a raised ring 75, a portion (e.g., a semi-circular portion) of which is positioned on top of the partial member 74, and a portion (e.g., a semi-circular portion) of which extends radially inwardly (away from the arcuate wall 72) from the spaced free ends 74a, 74b (only 74a shown). The raised ring member defines an aperture 78. The other of the first and second arms has a partial ring member 76 terminating in spaced free ends 76a, 76b and a semi-circular portion 77 coplanar with the partial ring member 76 but having a smaller circumference. The semi-circular portion 77 defines, with its arm member, an aperture 79. Preferably the apertures 78 and 79 are linearly aligned. In some embodiments, the interior wall of each of the arcuate walls 72 includes a groove configured to receive a seal 81, such as a gasket, O-ring or the like. This seal prevents leakage from the fluid channel or bore 35 when the core 13 is in a closed positioned, such as after fluid transfer has taken place and the bore 35 is moved out of fluid communication with the first and second coupling members.

In certain embodiments, the device 10 may be assembled by inserting two stored gates 30 into respective gate-receiving regions of the core 13, such as regions 51a and 51c in the clockwise direction from radially outwardly extending members 40, 42. Each arcuate housing member 70 is then positioned about the core 13 such that the free ends 74a, 74b of a first of the arcuate housing members 70 abut the free ends 76a, 76b of a second of the arcuate housing members 70, and the free ends 76a, 76b of the first arcuate housing member abut the free ends 74a, 74b of the second housing member. Each actuator 16, 17 is keyed into a respective top or bottom of the core 13 and fastened to the core such as with a suitable fastener 9.

Each of the connectors 12, 14, each carrying a slidable gate 30 positioned in the groove 25 of the connectors 12, 14, now may be coupled to the device 10. In some embodiments, the coupling is carried out by snapping the tabs 28a, 28b over the rims 50 of the core 13. The core 13 may then rotated. A spring plunger (not shown) or the like may be present to lock the device in this position until the spring plunger of the like is further actuated to release it.

This actuation of the core 13 relative to the housing members 70 also causes the first radially outwardly extending region 40 and the second radially outwardly extending region 42 of the core 13 to slide the connectors away from their respective gates 30 as well as align the bore 35 with the connectors, thereby establishing fluid communication between the connectors through the bore 35. That is, due to the presence of the radially outwardly extending regions 40 and 42, as the connectors 12, 14 are moved into a fluid transfer position in fluid communication with bore 35, the gates 30 that were respectively positioned in the connectors 12, 14 when the connectors were attached to the core 13 are prevented from accompanying them to the fluid transfer position by being obstructed by the radially outwardly extending regions 40 and 42. The gates 30 that slide out of the connectors thus remain in respective gate-receiving regions 51b, 51d of the core 13, while the connectors 12, 14 respectively engage one of the radially outwardly protruding regions 40, 42.

Upon completion of fluid transfer, further actuation of the core 13 relative to the housing members 17 slides the sterile stored gates 30 occupying gate-receiving regions 51a, 51c into respective grooves 25 of the connectors 12, 14 and also moves the bore 35 into a closed position out of alignment with the connectors 12, 14 and into sealing relation with the seals 81 to minimize or prevent fluid in the bore 35 from leaking out of the device 10. The connectors 12, 14, which each now hold a sterile gate 30 that had previously been stored in a gate-receiving region (e.g., 51a, 51c) of the core 13, can then be removed from the device 10 and optionally reused. In this decoupling position, the tabs 28a, 28b respectively align with the spaced slots 41a, 41b and 44a, 44b slots to facilitate their decoupling from the core 13. The device 10, in which the gates that were originally held by the connectors 12, 14 when the connectors were coupled to the core are trapped in the housing, can be properly disposed of.

In some embodiments, each of the above incremental rotations, e.g. to lock the connectors to the core and align them with the bore, and to slide the sterile stored gates 30 occupying gate-receiving regions 51a, 51c into respective grooves 25 of the connectors 12, 14 and move the bore 35 into a closed position out of alignment with the connectors, is preferably a 60° rotation. A feature (not shown) internal to the housing may be provided that interferes with the core and prevents over-rotation. Similarly, a feature (not shown) such as a saw-toothed element may be provided between the housing and the core that allows rotation in only one direction.

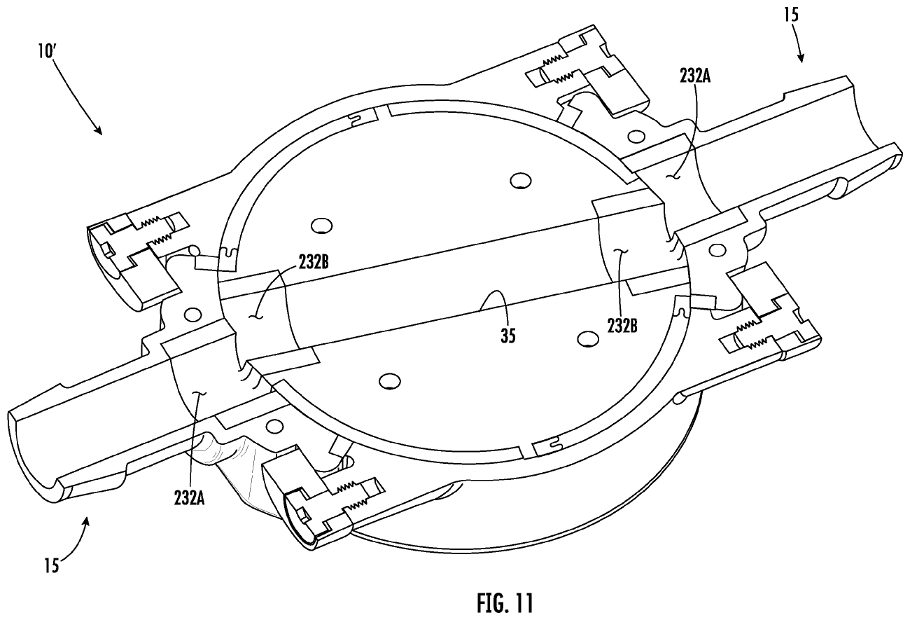

FIG. 11 illustrates a modified embodiment of device 10′ shown in an intermediate position and where the sealing mechanism is different. Specifically, in the embodiment of FIG. 11, a first seal 232A is positioned in the connector 15 as shown, and a second seal 232B is positioned at an end of the bore 35 and is rotatable with the core 13. A similar arrangement is present in the opposite connector 15 and opposite end of the bore 35.

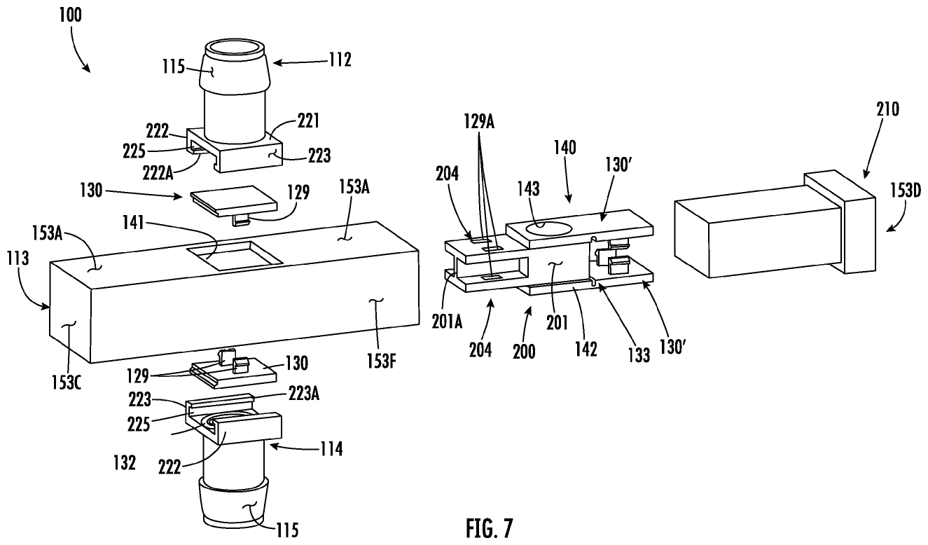

FIGS. 7, 8, 9A, 9B, 9C and 10 illustrate a linear embodiment of the fluid transfer device 100. In the embodiment shown, the fluid transfer device 100 includes a first coupling member or connector 112 attachable to a housing 113, and a second coupling member or connector 114 attachable to the housing 113. Each of the first and second coupling members 112 and 114 may be attached to or integral to a barbed fitting 115 or tri-clover fitting or the like, configured to connect to suitable tubing or the like in a conventional and well-known manner.

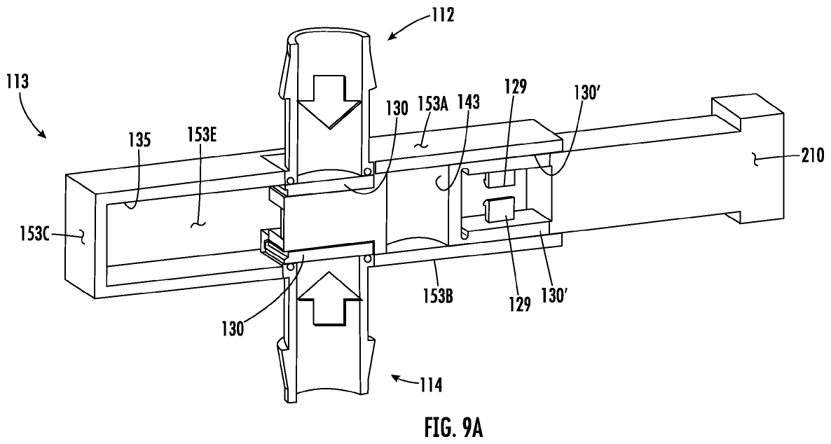

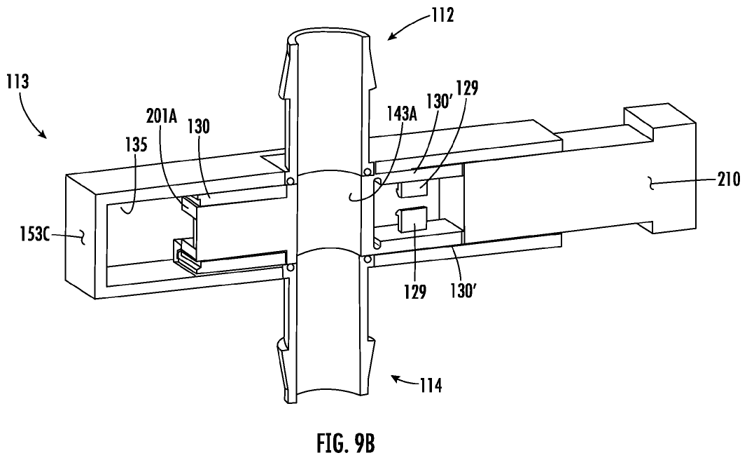

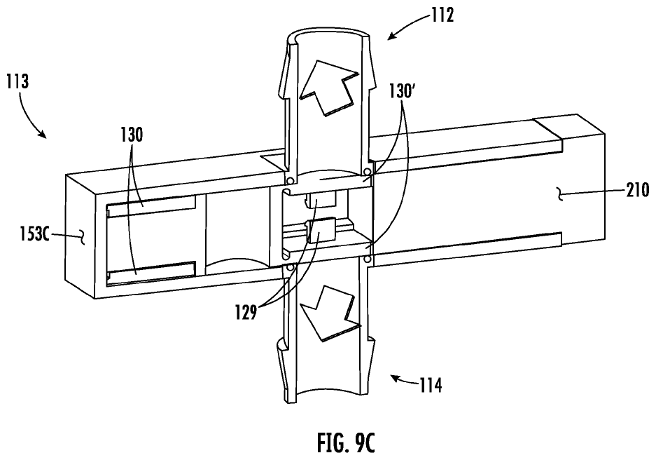

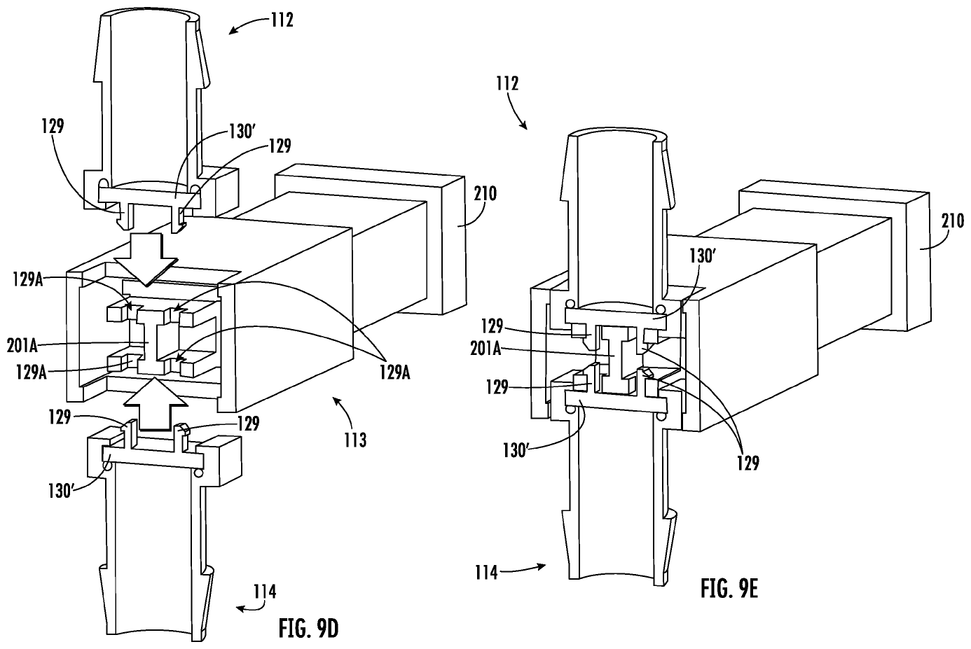

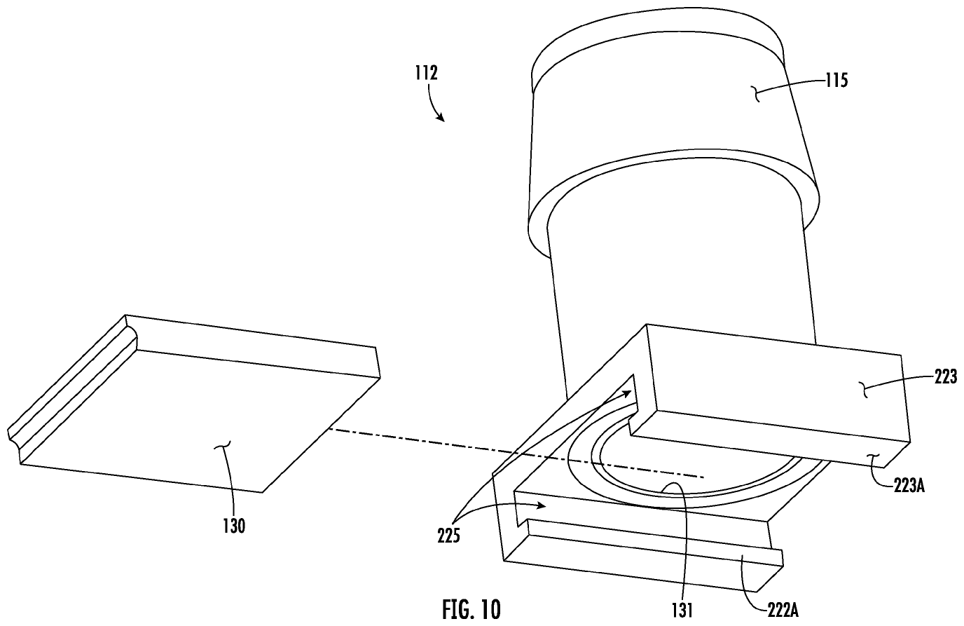

In certain embodiments, the first and second coupling members or connectors 112, 114 may be identical. The coupling member 112, 114 includes a generally flat outer surface 221, a first arm 222 and a second arm 223 spaced from the first arm, each extending in the same downwardly direction, away from the generally flat outer surface 221 as shown. As best seen in FIG. 10, the underside or bottom of each arm 222, 223 terminates in a respective inwardly extending flange 222A, 223A terminating in a free end so as to form, in cooperation with a respective arm 222, 223, a groove 225 configured to slidingly receive and hold a displaceable gate 130. Each groove 225 may be linear and dimensioned to receive a displaceable gate 130. Each coupling member 112, 114 has a through hole 131, and a groove for receiving a seal member 132 such as a gasket or O-ring to help seal a gate 130 against the through hole when the gate 130 is positioned in the groove 125. In some embodiments, each of the coupling members 112, 114 is symmetrical about a horizontal axis, and thus can be attached to the fluid transfer device in the orientation shown in FIG. 7, or in an up-side-down orientation to that shown in FIG. 7, i.e., rotated 180°. In certain embodiments, one or more (two shown) tabs or tab-like features 129 may extend from the gates 130 to secure the coupling members 112, 114 to the housing 113. The tabs 129 may be flexible to allow easy removability of the coupling members 112, 114 from the housing 113. In certain embodiments, the tabs 129 are configured to be received by corresponding apertures or slots 129A in the recessed regions 204 of the core 200 as best seen in FIGS. 9C, 9D and 9E.

In certain embodiments, the housing 113 is generally a rectangular box or parallelepiped, although other shapes may be used without departing from the spirit of this embodiment. It includes an internal cavity or channel 135 (FIG. 9A) that holds a valve core that enables fluid communication between the first and second coupling members 112, 114 when the first and second coupling members are sealingly engaged with the housing 113. A first wall 153A and an opposite second wall 153B of the housing 113 each include a respective through hole 141 (only one shown in FIG. 7) Preferably the through holes 141 are spaced 180° from each other and are of the same shape and size. The housing 113 may include opposite side walls 153C, 153D, with side wall 153D being provided by an actuator (e.g., plunger 210) in the embodiment shown. The housing 113 also may include opposite front and back walls 153E, 153F so that the internal cavity or channel 135 is closed except for the through holes 141. The housing 113 may be single-use (e.g., disposable) or may be re-used.

FIG. 7 also illustrates a valve core or carriage 200 in accordance with certain embodiments. The core 200 is preferably single-use. The core 200 is configured to be received in the cavity 135 and be slidable therein. Actuation of the core 200 may be carried out by any suitable means, such as with a plunger 210 or the like. Actuation may be manual or automatic. In certain embodiments, the core 200 includes a main body 201 that includes a leading edge 201A configured to engage and move gates 130 held by coupling members 112, 114 as discussed in greater detail below. In certain embodiments, the leading edge 201A may have an I-shape cross-section as best seen in FIG. 7, with the top or upper region of the “I” positioned to engage the gate 130 held in coupling member 112 when coupling member 112 is attached to the housing 113, and the bottom or lower region of the “I” positioned to engage the gate 130 held in the coupling member 114 when coupling member 114 is attached to the housing 113, upon actuation of the core 200.

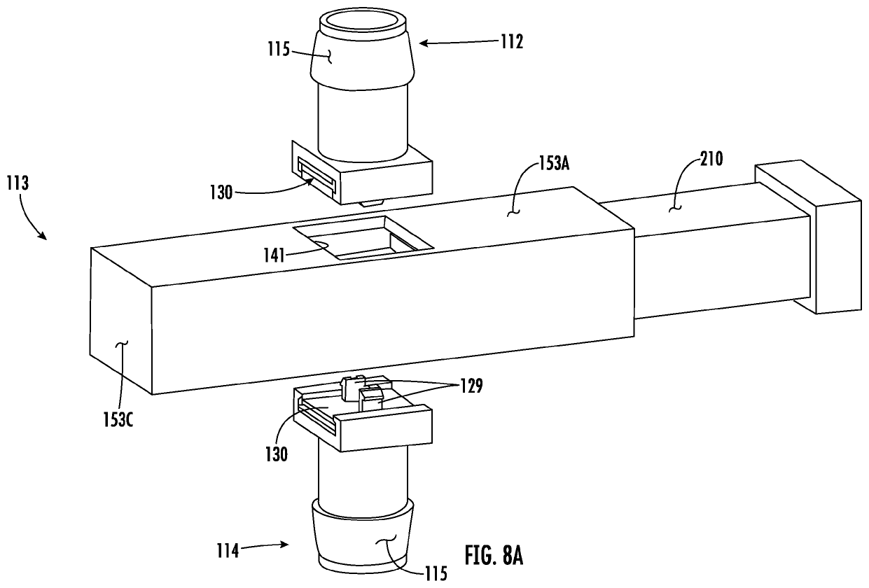

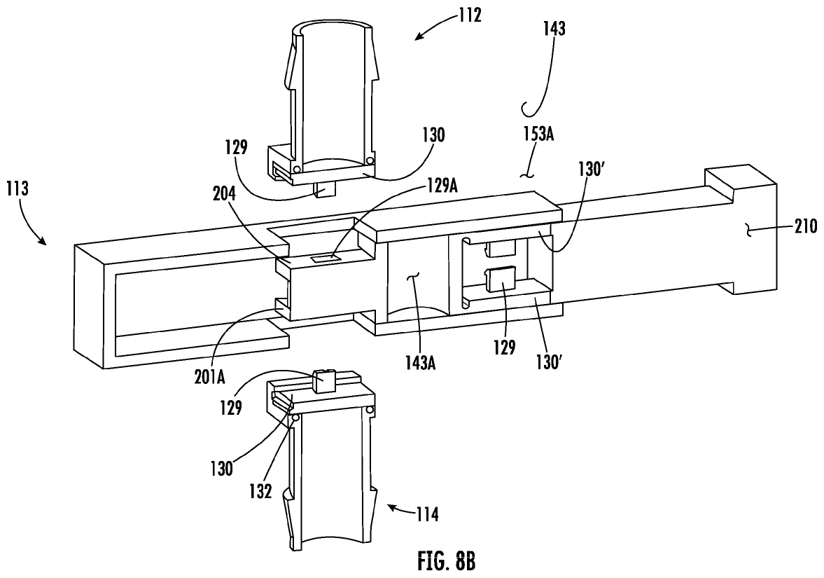

Integral to or coupled to the main body 201 are first axially or upwardly extending region 140 and second axially or downwardly extending region 142 (as depicted in FIG. 7). Each of the first and second axially extending regions 140, 142 is generally flat or planar and includes a respective through-hole 143 in fluid communication with an inner bore 143A (FIGS. 8B, 9B). Preferably the through holes 143 are spaced 180° from each other. Each of the first and second axially extending regions 140, 142 define, with the main body 201, opposite recess regions 204 configured to receive and accommodate a gate 130. In some embodiments, each of the first and second axially extending regions 140, 142 has the same or similar dimensions as a gate 130 (except for the presence of the through hole 143).

Removably attached to the axially extending regions 140, 142 are respective substitute or stored gates 130′. A thin or brittle section 133 between each gate and the axially extending regions 140, 142 may be used to facilitate removability or break-away of the gates 130′, such as by manually or automatically flexing, twisting or snapping them off. Suitable materials for the stored gates that enable the break-away feature include rigid amorphous plastics such as polyethersulphone.

FIGS. 9A, 9B and 9C illustrate operation of the device in accordance with certain embodiments. FIG. 9A shows the coupling members 112, 114, with pre-installed gates 130 (or gates from a previous connection) held by the coupling members 112, 114, inserted into the housing 113. In this position, the gates 130 occupy the recessed regions 204 (effectively gate-receiving regions) of the core 200 and block fluid communication from the coupling members 112, 114 to the cavity or channel 135 and between the coupling members 112, 114.

FIG. 9B illustrates actuation of the assembly with the plunger 210, which urges the core 200 towards side wall 153C, whereby the leading edge 201A engages the gates 130 held in respective coupling members 112, 114 and pushes them out of engagement with their respective coupling members and into or onto the respective recessed regions 204 of the core. Consequently, fluid communication between the coupling members 112, 114 and the cavity 135, and between the coupling members 112, 114 themselves, is established via the inner bore 143A, as gates 130 are no longer blocking the through-hole 143 and access to inner bore 143A in the cavity 135.

Upon completion of fluid transfer through inner bore 143A, further actuation of the plunger 210 (FIG. 9C) relative to the housing 113 slides the core 200 further towards side wall 153C, positioning the substitute or stored gates 130′ in respective coupling members 112, 114, thereby again blocking fluid communication with the cavity 135 to close the fluid path between coupling members. Subsequent removal of the coupling members from the housing 113, such as by a twisting or pivoting action, snaps off or disconnects the stored gates 130′ from their respective axially extending regions 140, 142, allowing removal of the coupling members 112, 114 from the housing 130′, each now carrying a stored gate 130′. Thus the coupling members 112, 114, which each now hold a sterile gate 130′ that had previously been stored, can be optionally reused. The housing 113 and core 200 may be properly disposed of.

In some embodiments, the device may be supplied to the end user with a tear-away outer seal to maintain sterility of the device. In some embodiments, the device may also be double-bagged and sterilized to maintain sterility during shipment and storage.

Sterilization may be carried out by conventional means, including by gamma radiation, X-ray, autoclaving, steaming, ethylene oxide treatment. Herein, “aseptic” is defined as free or substantially free from contamination caused by harmful bacteria, viruses, or other microorganisms, such as a level of asepsis below about 1 CFU/ml.

Suitable materials of construction include materials capable of withstanding the conditions typically encountered by such devices, including those of sterilization. Suitable materials include but are not limited to plastic, stainless steel and aluminum. Suitable plastic materials may include but are not limited to polysulfone, glass filled polysulfone, polyphenylene sulfide, glass filled polyphenylene sulfide, polyphenyl sulfone and glass filled polyphenyl sulfone are all acceptable materials due to their biocompatibility, chemical, heat and creep resistance. The plastic components may be formed by machining or molding. The seals used in the embodiments disclosed herein can be made of but not limited to silicone, rubber, including natural and synthetic rubbers, thermoplastic elastomers, polyolefins, polytetrafluoroethylene (PTFE) and PTFE blends, thermoplastic perfluoropolymer resins, urethanes, EPDM rubber, and other polymeric materials. Fluids to be transferred include liquids and gases.

The term “radial” means perpendicular to an axis, or in the direction of a radius of a circular or cylindrical element or configuration, such as perpendicular to the longitudinal axis of the core 13.

Reference throughout this specification to “one embodiment,” “certain embodiments,” “one or more embodiments,” “some embodiments,” or “an embodiment” indicates that a feature, structure, material, or characteristic described in connection with the embodiment is included in at least one embodiment of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment. Nonetheless, it is to be understood that any feature described herein can be incorporated within any embodiment(s) disclosed herein.

Publications of patent applications and patents and other non-patent references cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. A fluid transfer device, comprising: a housing; a core positioned in said housing for relative movement with said housing, the core having a bore having an inlet and an outlet spaced from the inlet, and first and second gate-receiving regions spaced from said inlet and said outlet; and an actuator operatively connected in said device to cause relative movement between said core and said housing.

2. The fluid transfer device of claim 1, further comprising first and second gates respectively positioned in said first and second gate-receiving regions.

3. The fluid transfer device of claim 2, further comprising a first connector and a second connector, said first connector comprising a third gate displaceable from said first connector, said second connector comprising a fourth gate displaceable from said second connector, said first connector being attached or attachable to said core, and said second connector being attached or attachable to said core.

4. The fluid transfer device of claim 3, wherein said first gate is movable from a stored position in said first gate-receiving region to a position in said first connector.

5. The fluid transfer device of claim 3, wherein said second gate is movable from a stored position in said second gate-receiving region to a position in said second connector.

6. The fluid transfer device of claim 2, wherein said core is disk-shaped, said first and second gate-receiving regions have a radius of curvature, and wherein said first and second gates have a radius of curvature that matches the radius of curvature of said gate-receiving regions.

7. The fluid transfer device of claim 3, wherein said first connector has a groove configured to slidingly receive said third gate.

8. The fluid transfer device of claim 7, wherein said core has a radially outwardly protruding region configured to be received by said groove.

9. The fluid transfer device of claim 1, wherein said relative movement is linear movement.

10. The fluid transfer device of claim 3, wherein said core comprises a first region configured to receive said third gate when said third gate is displaced from said first connector, and a second region configured to receive said fourth gate when said fourth gate is displaced from said second connector.

11. The fluid transfer device of claim 3, wherein said first and second gates are removably coupled to said core.

12. A method of creating an aseptic connection, comprising coupling a first connector and a second connector to a valve core, the valve core having an inner bore, first and second gate-receiving regions and first and second gates respectively occupying said first and second gate-receiving regions; actuating said valve core to establish fluid communication between said first and second connectors through said inner bore; and further actuating said valve core to move said first connector into engagement with said first gate, and to move said second connector into engagement with said second gate.

13. The method of claim 12, further comprising removing said first and second connectors from said valve core after said first and second gates are respectively engaged in said first and second connectors.

14. The method of claim 12, wherein said valve core comprises first and second radially outwardly protruding regions, and wherein said first and second radially outwardly protruding regions respectively receive said first and second connectors when said valve core is actuated to establish fluid communication between said first and second connectors through said inner bore.

15. The method of claim 14, wherein said first and second connectors each hold a displaceable gate when said first and second connectors are coupled to said valve core, and wherein said first and second radially outwardly protruding regions respectively displace said first and second displaceable gates from said first and second connectors, respectively, when said valve core is actuated to establish fluid communication between said first and second connectors through said inner bore.
